(19) United States
(12) Patent Application Publication
(10) Pub. No.: US 2008/0069892 A1
(43) Pub. Date: Mar. 20, 2008

(54) Powder Formulations

(75) Inventors: Karl Reizlein, Monheim (DE); Joachim Kruger, Monheim (DE); Hanns-Peter Muller, Odenthal (DE); Horst Gruttmann, Leverkusen (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AKTIENGESELLSCHAFT, Monheim (DE)

(21) Appl. No.: 10/587,143

(22) PCT Filed: Jan. 19, 2005
(86) PCT No.: PCT/EP05/00474

§ 371 Date: Apr. 26, 2007

(30) Foreign Application Priority Data
Jan. 28, 2004 (DE) 102004004143.1

(51) Int. Cl. A61K 9/14

(52) U.S. Cl. 424/501

(57) ABSTRACT

The present invention relates to powder formulations which consist of at least one agrochemical active substance, at least one polyurethane and/or polyurethane urea, and, if appropriate, additives and which have a particle diameter of less than 125 μm, to processes for the preparation of these formulations and to their use for applying agrochemical active substances.

The present invention relates to new powder formulations comprising agrochemical active substances and polymers, to a process for the preparation of these formulations, and to their use for applying agrochemical active substances.

Microparticles comprising specific polymers as a mixture with agrochemical active substances are already known from WO 99/00013. These preparations are made by dissolving polymers and agrochemical active substances in an organic solvent which is sparingly miscible with water, then dispersing this solution in water using emulsifiers, thereupon evaporating the solvent, and separating the resulting microparticles from the aqueous phase by decanting and/or filtration and subsequently drying them. However, the disadvantage of this process is that a large number of complicated procedures have to be carried out and thus the use of relatively large apparatuses is necessary when carrying out the process on an industrial scale.

U.S. Pat. No. 4,400,374 describes compositions for the controlled release of agrochemical active substances in an aqueous environment which comprise a polymer, in particular a thermoplastic material, and a porosity-inducing agent.

WO 03/056921 and WO 03/105584 describe powder formulations which comprise biodegradable hydroxyl-containing polyesters or copolymers of styrene and acrylonitrile.

There have now been found new powder formulations consisting of
at least one agrochemical active substance,
at least one polyurethane and/or polyurethane urea and,
if appropriate, additives
   and having a particle diameter of less than 125 μm.

It has furthermore been found that the powder formulations according to the invention can be prepared by homogenizing a mixture of
at least one agrochemical active substance,
at least one polyurethane and/or polyurethane urea and,
if appropriate, additives
   in the melt at temperatures between 50° C. and 200° C. and, when cold, comminuting the mixture in such a way that a powder is obtained in which the particles have a diameter of less than 125 μm.

Finally, it has been found that the powder formulations according to the invention are highly suitable for applying the agrochemical active substances which they contain to plants and/or their environment.

It is surprising that the powder formulations according to the invention are more suitable for applying the agrochemical active substances which they contain than the prior-art preparations of the most similar constitution. What is particularly unexpected is that the active components are released in the specifically desired amount over a prolonged period.

Furthermore, it had to be assumed that carrying out the process according to the invention would, after cooling of the melt, result in soft resins which are sticky at room temperature since mixtures of polyurethane and/or polyurethane urea and agrochemical active substances generally have low melting points. However, in contrast to what was expected, products are obtained which are so brittle that they can be comminuted with the aid of customary mills without additional cooling to give free-flowing powders which do not form aggregates.

The powder formulations according to the invention contain one or more agrochemical active substances.

Agrochemical active substances are understood as meaning, for the present purpose, all substances which are customary for the treatment of plants. Substances which may preferably be mentioned are flugicides, bactericides, insecticides, acaricides, nematicides, herbicides, plant growth regulators, plant nutrients and repellents. Solid agrochemical active substances are preferred.

Examples of fungicides which may be mentioned are:

2-anilino-4-methyl-6-cyclopropylpyrimidine; 2′,6′-dibromo-2-methyl-4′-trifluoromethoxy-4′-trifluoromethyl-1,3-thiazole-5-carboxanilide; 2,6-dichloro-N-(4-trifluoromethylbenzyl)benzamide; (E)-2-methoximino-N-methyl-2-(2-phenoxyphenyl)acetamide; 8-hydroxyquinoline sulfate; methyl (E)-2-{2-[6-(2-cyanophenoxy)pyrimidin-4-yloxy]phenyl}-3-methoxyacrylate; methyl (E)-methoximino[alpha-(o-tolyloxy)-o-tolyl]acetate; 2-phenylphenol (OPP), aldimorph, ampropylfos, anilazin, azaconazole,

benalaxyl, benodanil, benomyl, binapacryl, biphenyl, bitertanol, blasticidin-S, bromuconazole, bupirimate, buthiobate,

calcium polysulfide, captafol, captan, carbendazim, carboxin, quinomethionate, chloroneb, chloropicrin, chlorothalonil, chlozolinate, cufraneb, cymoxanil, cyproconazole, cyprofuram, carpropamid,

dichlorophen, diclobutrazol, dichlofluanid, diclomezine, dicloran, diethofencarb, difenoconazole, dimethirimol, dimethomorph, diniconazole, dinocap, diphenylamine, dipyrithion, ditalimfos, dithianon, dodine, drazoxolon,

edifenphos, epoxyconazole, ethirimol, etridiazole,

fenarimol, fenbuconazole, fenfuram, fenitropan, fenpiclonil, fenpropidin, fenpropimorph, fentin acetate, fentin hydroxide, ferbam, ferimzone, fluazinam, fludioxonil, fluoromide, fluquinconazole, flusilazole, flusulfamide, flutolanil, flutriafol, folpet, fosetyl-aluminum, fthalide, fuberidazol, furalaxyl, furmecyclox, fenhexamid,

guazatine,

hexachlorobenzene, hexaconazole, hymexazol,

imazalil, imibenconazole, iminoctadine, iprobenfos (IBP), iprodione, isoprothiolan, iprovalicarb,

kasugamycin, copper preparations such as: copper hydroxide, copper naphthenate, copper oxychloride, copper sulfate, copper oxide, oxine-copper and Bordeaux mixture,

mancopper, mancozeb, maneb, mepanipyrim, mepronil, metalaxyl, metconazole, methasulfocarb, methfuroxam, metiram, metsulfovax, myclobutanil,

nickel dimethyldithiocarbamate, nitrothal-isopropyl, nuarimol,

ofurace, oxadixyl, oxamocarb, oxycarboxin,

pefurazoate, penconazole, pencycuron, phosdiphen, pimaricin, piperalin, polyoxin, probenazole, prochloraz, procymidon, propamocarb, propiconazole, propineb, pyrazophos, pyrifenox, pyrimethanil, pyroquilon,

quintozene (PCNB), quinoxyfen,

sulfur and sulfur preparations, spiroxamine,

tebuconazole, tecloftalam, tecnazene, tetraconazole, thiabendazole, thicyofen, thiophanate-methyl, thiram, tolclophos-methyl, tolylfluanid, triadimefon, triadimenol, triazoxide, trichlamide, tricyclazole, tridemorph, triflumizole, triforine, triticonazole, trifloxystrobin,

validamycin A, vinclozolin,

zineb, ziram, and

2-[2-(1-chlorocyclopropyl)-3-(2-chlorophenyl)-2-hydroxypropyl]-2,4-dihydro-[1,2,4]-triazole-3-thione.

Examples of bactericides which may be mentioned are:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Examples of insecticides, acaricides and nematicides which may be mentioned are:

abamectin, acephate, acrinathrin, alanycarb, aldicarb, alphamethrin, amitraz, avermectin, AZ 60541, azadirachtin, azinphos A, azinphos M, azocyclotin,

Bacillus thuringiensis, 4-bromo-2-(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, BPMC, brofenprox, bromophos A, bufencarb, buprofezin, butocarboxin, butylpyridaben,

cadusafos, carbaryl, carbofuran, carbophenothion, carbosulfan, cartap, chloethocarb, chloretoxyfos, chlorfenvinphos, chlorfluazuron, chlormephos, N-[(6-chloro-3-pyridinyl)-methyl]-N′-cyano-N-methyl-ethanimidamide, chlorpyrifos, chlorpyrifos M, cis-resmethrin, clocythrin, clofentezine, clothianidin, cyanophos, cycloprothrin, cyfluthrin, cyhalothrin, cyhexatin, cypermethrin, cyromazine,

deltamethrin, demeton-M, demeton-S, demeton-S-methyl, diafenthiuron, diazinon, dichlofenthion, dichlorvos, dicliphos, dicrotophos, diethion, diflubenzuron, dimethoate,

dimethylvinphos, dioxathion, disulfoton,

emamectin, esfenvalerate, ethiofencarb, ethion, ethofenprox, ethoprophos, etrimphos,

fenamiphos, fenazaquin, fenbutatin oxide, fenitrothion, fenobucarb, fenothiocarb, fenoxycarb, fenpropathrin, fenpyrad, fenpyroximate, fenthion, fenvalerate, fipronil, fluazuron, flucycloxuron, flucythrinate, flufenoxuron, flufenprox, fluvalinate, fonophos, formothion, fosthiazate, fubfenprox, furathiocarb,

HCH, heptenophos, hexaflumuron, hexythiazox,

imidacloprid, iprobenfos, isazophos, isofenphos, isoprocarb, isoxathion, ivermectin,

lambda-cyhalothrin, lufenuron,

malathion, mecarbam, mevinphos, mesulfenphos, metaldehyde, methacrifos, methamidophos, methidathion, methiocarb, methomyl, metolcarb, milbemectin, monocrotophos, moxidectin,

naled, NC 184, nitenpyram,

omethoate, oxamyl, oxydemethon M, oxydeprofos,

parathion A, parathion M, permethrin, phenthoate, phorate, phosalone, phosmet, phosphamidon, phoxim, pirimicarb, pirimiphos M, pirimiphos A, profenophos, promecarb, propaphos, propoxur, prothiophos, prothoate, pymetrozin, pyrachlophos, pyridaphenthion, pyresmethrin, pyrethrum, pyridaben, pyrimidifen, pyriproxifen,

quinalphos,

salithion, sebufos, silafluofen, sulfotep, sulprofos,

tebufenozide, tebufenpyrad, tebupirimiphos, teflubenzuron, tefluthrin, temephos, terbam, terbufos, tetrachlorvinphos, thiacloprid, thiafenox, thiamethoxam, thiodicarb, thiofanox, thiomethon, thionazin, thuringiensin, tralomethrin, transfluthrin, triarathen, triazophos, triazuron, trichlorfon, triflumuron, trimethacarb,

vamidothion, XMC, xylylcarb, zetamethrin.

Examples of herbicides which may be mentioned are:

anilides such as, for example, diflufenican and propanil; arylcarboxylic acids, such as, for example, dichloropicolinic acid, dicamba and picloram; aryloxyalkanoic acids such as, for example, 2,4-D, 2,4-DB, 2,4-DP, fluroxypyr, MCPA, MCPP and triclopyr, aryloxyphenoxyalkanoic esters such as, for example, diclofop-methyl, fenoxaprop-ethyl, fluazifop-butyl, haloxyfop-methyl and quizalofop-ethyl; azinones such as, for example, chloridazon and norflurazon; carbamates such as, for example, chlorpropham, desmedipham, phenmedipham and propham; chloroacetanilides such as, for example, alachlor, acetochlor, butachlor, metazachlor, metolachlor, pretilachlor and propachlor; dinitroanilines such as, for example, oryzalin, pendimethalin and trifluralin; diphenyl ethers such as, for example, acifluorfen, bifenox, fluoroglycofen, fomesafen, halosafen, lactofen and oxyfluorfen; ureas such as, for example, chlortoluron, diuron, fluometuron, isoproturon, linuron and methabenzthiazuron; hydroxylamines such as, for example, alloxydim, clethodim, cycloxydim, sethoxydim and tralkoxydim; imidazolinones such as, for example, imazethapyr, imazamethabenz, imazapyr and imazaquin; nitriles such as, for example, bromoxynil, dichlobenil and ioxynil; oxyacetamides such as, for example, mefenacet; sulfonylureas such as, for example, amidosulfuron, bensulfuron-methyl, chlorimuron-ethyl, chlorsulfuron, cinosulfuron, metsulfuron-methyl, nicosulfuiron, primisulfuron, pyrazosulfuron-ethyl, thifensulfuron-methyl, triasulfuron and tribenuron-methyl; thiocarbamates such as, for example, butylate, cycloate, di-allate, EPTC, esprocarb, molinate, prosulfocarb, thiobencarb and tri-allate; triazines such as, for example, atrazine, cyanazine, sirnazine, simetryne, terbutryne and terbutylazine; triazinones such as, for example, hexazinon, metamitron and metribuzin; others such as, for example, aminotriazole, benfuresate, bentazone, cinmethylin, clomazone, clopyralid, difenzoquat, dithiopyr, ethofumesate, fluorochloridone, glufosinate, glyphosate, isoxaben, pyridate, quinchlorac, quinmerac, sulphosate and tridiphane. Others which may be mentioned are 4-amino-N-(1,1-dimethylethyl)-4,5-dihydro-3-(1-methylethyl)-5-oxo-1H-1,2,4-triazole-1-carboxamide and methyl 2-((((4,5-dihydro-4-methyl-5-oxo-3-propoxy-1H-1,2,4-triazol-1-yl)carbonyl)amino)sulfonyl)benzoate.

Examples of plant growth regulators which may be mentioned are chlorcholin chloride and ethephon.

Examples of plant nutrients which may be mentioned are customary inorganic or organic fertilizers for providing plants with macro- and/or micronutrients.

Examples of repellents which may be mentioned are diethyltoluamide, ethylhexanediol and butopyronoxyl.

Examples which may be mentioned of insecticides which may preferably be present in the powder formulations according to the invention are the following active substances:

imidacloprid, thiacloprid, thiamethoxam, acetamiprid, clothianidin, betacyfluthrin, cypermethrin, transfluthrin, lambda-cyhalothrin and azinphos-methyl.

Examples which may be mentioned of herbicides which can preferably be present in the powder formulations according to the invention are the following active substances:

propoxycarbazone-sodium, flucarbazone-sodium, amicarbazone, dichlobenil and phenyluracils.

The powder formulations according to the invention comprise polyurethane(s) and/or polyurethane urea(s), if appropriate in admixture with one or more further polymers.

Polyurethanes or polyurethane ureas which can be used for the preparation of the powder formulations according to the invention are reaction products of aliphatic or aromatic diisocyanates with macropolyols, macropolyamines, chain extenders and, if appropriate, chain terminators.

Macropolyols and macroamines are understood as meaning hydroxyl- and amine-comprising compounds such as, for example, polyesters, polycarbonates and polyethers having a molar mass of more than 400 g/mol.

Chain extenders are understood as meaning short-chain diols, alkanolamines, water and diamines with a molar mass of less than 400 g/mol.

Chain terminators are understood as meaning monoalcohols, monoamines and ammonia.

The ratio of the isocyanate groups to isocyanate-reactive groups is 1.1:1 to 1:2. In order to achieve the desired characteristics such as millability and release of the active substance, the starting materials can be combined with one another as desired. As a rule, difunctional compounds are used.

However, it is also possible to employ such an amount of mono- or higher-functional compounds that thermoplastic processing in the melt or else in solution is possible.

The polyurethanes or polyurethane ureas which can be used for the preparation of the powder formulations according to the invention preferably comprise urethane groups (NH—CO—O) or urea groups (NH—CO—NH) in an amount of from 1 to 55% by weight, especially preferably from 1.5 to 50% by weight.

The following may be employed as diisocyanates: ethylene diisocyanate, 1,4-tetramethylene diisocyanate, 1,6-hexamethylene diisocyanate, 1,12-dodecanediisocyanate, cyclobutanediisocyanate, cyclohexane-1,3- and -1,4-diisocyanate, and any desired mixtures of these isomers; 1-isocyanato-3,3,5-trimethyl-5-isocyanatomethylcyclohexane, 2,4- and 2,6-hexahydrotoluylenediisocyanate and any desired mixtures of these isomers, hexahydro-1,3-and/or -1,4-phenylenediisocyanate, perhydro-2,4′- and/or 4,4′-diphenylmethanediisocyanate and norbornane diisocyanate (for example U.S. Pat. No. 3,492,330). The isomers and isomer mixtures of toluylene diisocyanate (TDI), naphthylene diisocyanate, diphenylmethanediisocyanate (MDI), phenylenediisocyanate and xylylenediisocyanate. Preferred in addition to 1,6-hexamethylenediisocyanate (HDI) are 1-isocyanate-3,3,5-trimethyl-5-isocyanatomethylcyclohexane (IPDI) and perhydro-2,4′- and/or 4,4′-diphenylmethanediisocyanates (H₁₂ MDI). Furthermore preferred are toluylenediisocyanate (TDI) and diphenylmethanediisocyanate (MDI).

If appropriate, higher-functional isocyanates such as, for example, the isocyanurate of HDI (Desmodur® N 3300, Bayer) or the trimer of IPDI (Desmodur® Z 4300, Bayer) can also be used concomitantly. However, care must be taken that a mean functionality of two is not exceeded substantially. If appropriate, reactants with a higher finctionality must be compensated for by the concomitant use of other reactants with a functionality of less than two. Monofunctional isocyanates which are suitable for this purpose are, for example, stearyl isocyanate and cyclohexyl isocyanate.

Examples of suitable polyesters are reaction products of polyhydric, preferably dihydric and if appropriate additionally trihydric alcohols with polybasic, preferably dibasic, carboxylic acids or their esterifiable derivatives. The polycarboxylic acids can be aliphatic, cycloaliphatic, aromatic and/or heterocyclic in nature, for example they can be substituted by halogen atoms and/or unsaturated.

Examples of such carboxylic acids and their derivatives which may be mentioned are:

Succinic acid, adipic acid, phthalic acid, isophthalic acid, phthalic anhydride, tetrahydrophthalic anhydride, hexahydrophthalic anhydride, tetrachlorophthalic anhydride, endomethylenetetrahydrophthalic anhydride, maleic anhydride, fumaric acid, dimerized and trimerized unsaturated fatty acids, if appropriate as a mixture with monomeric unsaturated fatty acids, dimethyl terephthalate and terephthalic acid bisglycol ester.

Examples of suitable polyhydric alcohols are ethylene glycol, propylene 1,2-glycol, propylene 1,3-glycol, butylene 1,4-glycol, butylene 2,3-glycol, hexane-1,6-diol, octane-1,8-diol, neopentyl glycol, 1,4-bis(hydroxymethyl)cyclohexane, trimethylolpropane, trimethylolethane, furthermore di-, tri-, tetra- and higher polyethylene glycols, di- and higher polypropylene glycols and di- and higher polybutylene glycols. The polyesters can have terminal carboxyl groups in proportion. Polyesters of lactones, for example ε-caprolactone, or of hydroxycarboxylic acids, for example ω-hydroxycaproic acid, can also be employed. However, it is also possible to employ the hydroxyl-functional polyesters which are known from fat chemistry, such as, for example, castor oil and its transesterification products.

Hydroxyl-containing polycarbonates which are suitable are those of the known type, for example those which can be prepared by reacting diols such as propane-1,3-diol, butane-1,4-diol and/or hexane-1,6-iol, diethylene glycol, triethylene glycol, tetraethylene glycol or thiodiglycol, with diaryl carbonates, for example diphenyl carbonate or phosgene (DE-A 16 94 080, DE-A 22 21 751).

In addition to the polyester polyols and the polycarbonate diols, it is also possible to employ mixtures of polyether polyols and polyester polyols and mixtures of polyether polyols and polycarbonate diols.

Suitable polyether diols can be prepared by reacting one or more alkylene oxides having 2 to 4 carbon atoms in the alkylene radical with a starter molecule which contains two bound active hydrogen atoms. Examples of alkylene oxides which may be mentioned are: ethylene oxide, 1,2-propylene oxide, epichlorohydrin and 1,2-butylene oxide and 2,3-butylene oxide. It is preferred to employ ethylene oxide, propylene oxide and mixtures of 1,2-propylene oxide and ethylene oxide. The alkylene oxides can be used individually in succession one after the other or else as mixtures. Examples of suitable starter molecules are: water, amino alcohols such as N-alkyldiethanolamine, for example N-methyldiethanolamine and diols, such as ethylene glycol, 1,3-propylene glycol, 1,4-butanediol and 1,6-hexanediol. If appropriate, mixtures of starter molecules may also be employed.

Other suitable polyether diols are the hydroxyl-containing polymerization products of tetrahydrofuran.

To keep low the plasticizing activity of the polyether diols, they should be used in subordinate amounts.

Chain extenders which are employed are short-chain diols and/or diamines with a molecular weight of from 60 to 400 g/mol, preferably aliphatic diols having 2 to 14 carbon atoms such as, for example, ethanediol, 1,6-hexanediol, diethylene glycol, dipropylene glycol and, in particular, 1,4-butanediol, or (cyclo)aliphatic diamines such as, for example, isophorone diamine, ethylene diamine, 1,2-propylene diamine, 1,3-propylene diamine, N-methylpropylene 1,3-diamine, N,N′-dimethylethylene diamine. Also suitable are diesters of terephthalic acid with glycols having 2 to 4 carbon atoms and hydroxyalkylene ethers of hydroquinone. Mixtures of the abovementioned chain extenders can also be employed. In addition, smaller amounts of triols may also be added. Especially preferred are 1,2-propanediol, 1,4-butanediol and 1,6-hexanediol.

Examples of alkanolamines are 2-aminoethanol and 2-methylaminoethanol.

Furthermore, customary monofunctional compounds can also be employed in small amounts, for example as chain terminators or as mold release aids.

Examples which may be mentioned are alcohols such as ethanol, butanol, octanol and stearyl alcohol or amines such as benzylamine, butylamine and stearylamine.

Polyurethanes and/or polyurethane ureas having a mean molar mass of from 200 to 50 000 glmol, preferably of from 250 to 20 000 g/mol, should be used for the preparation of the powder formulations according to the invention.

The polyurethanes which can be used for the preparation of the powder formulations according to the invention can be prepared by the prepolymer method, in which the diisocyanate/diisocyanate mixture is initially mixed with the polyol/polyol mixture and reacted, whereby a prepolymer is obtained, whereupon the chain extension is carried out continuously, if appropriate in solvent.

A further possible embodiment consists in metering the isocyanate-containing compound, if appropriate with catalysis, into the one or more compound/compounds which react(s) with isocyanates and which is/are present in a solvent, for example acetone, chloroform or methylene chloride. The reaction can be completed by continuing to heat, with stirring.

Additives which may be present in the plant treatment compositions according to the invention are all customary substances which can be employed in such polymer preparations. Those which are preferably suitable are fillers, lubricants which are known from polymer technology, glidants and stabilizers.

Examples of fillers which may be mentioned are: titanium dioxide, barium sulfate, furthermore aluminum oxides, silicas, clays, precipitated or colloidal silicon dioxide, and phosphates.

Examples of lubricants and glidants which may be mentioned are: magnesium stearate, stearic acid, talc and bentonites.

Suitable stabilizers are antioxidants and substances which protect the polymers from undesired degradation during processing.

The concentrations of individual components in the powder formulations according to the invention can be varied within a substantial range. Thus, the content
of agrochemical active substances is generally between 1 and 50% by weight, preferably between 5 and 40% by weight,
of polyurethane(s) and/or polyurea(s) is generally between 50 and 99% by weight, preferably between 55 and 95% by weight, and
of additives is generally between 0 and 30% by weight, preferably between 0 and 20% by weight.

The powder formulations can be prepared on extruders, kneaders or in stirred vessels. To this end, the polyurethanes and/or polyurethane ureas are mixed with the agrochemical active substances at 50 to 200° C. Any solvent which is present can be removed via an evaporation extruder or, from a stirred vessel, via a distillation head.

The resulting liquid and homogeneous mixture is transported to cooling belts or cooling drums with the aid of customary discharge devices. When cold, the solidified product is removed from the cooling device and crushed. The crude granules obtained are subsequently comminuted with customary grinding equipment and screened to give a powder in which the particles have a diameter of less than 125 μm.

A further possible embodiment consists in preparing a low-melting, low-molecular-weight polyurethane with good crystallization properties, dissolving therein, at elevated temperature, a further polyurethane and the agrochemical active substance, and comminuting the mixture which is solid and nontacky at room temperature. This embodiment is particularly suitable for active substances which already undergo decomposition at temperatures of from 80 to 150° C.

Suitable grinding equipments are all mills which are conventionally employed for such purposes. Mills which can preferably be used are pinned-disk mills, ball mills, jet mills or classifier mills, a mill which may be mentioned by way of example being an ACM 2 type mill from Hosokawa Mikropul.

The powder formulations according to the invention, as such or after addition of further formulation auxiliaries, can be employed for applying agrochemical active substances in crop protection in agriculture, forestry or horticulture. Formulation auxiliaries which are suitable in this context are all those components which can generally be used in plant treatment compositions such as, for example, colorants, wetters, dispersants, emulsifiers, antifoams, preservatives, humectant components, antifreeze agents, secondary thickeners, solvents, and, when seed-dressing products are prepared, also stickers.

Colorants which can be employed for the further processing of the powders according to the invention as plant treatment compositions are all those colorants which are customary for such purposes. Colorants which can be used are sparingly water-soluble pigments and also water-soluble dyes. Examples which may be mentioned are those known under the names Rhodamin B, C.I.Pigment Red 112 and C.I.Solvent Red 1.

Suitable wetters which can be used for formulating the powders according to the invention are all those materials which promote wetting and which are customary for the formulation of agrochemical active substances. Materials which can preferably be used are alkylnaphthalenesulfonates, such as diisopropylnaphthalenesulfonate or diisobutylnaphthalenesulfonate.

Dispersants and/or emulsifiers which are suitable for formulating the powders according to the invention are all those nonionic, anionic and cationic dispersants which are conventionally used in the formulation of agrochemical active substances. Materials which can preferably be used are nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristyrylphenol polyglycol ethers, and their phosphated or sulfated derivatives. Suitable anionic dispersants are, in particular, lignosulfonates, polyacrylic acid salts and arylsulfonate/formaldehyde condensates.

Antifoams which can be used for formulating the powders according to the invention are all those materials which inhibit foaming and which are customary for the formulation of agrochemical active substances. Materials which can preferably be used are silicone antifoams and magnesium stearate.

Preservatives which can be used for formulating the powders according to the invention are all those substances which are conventionally used for such purposes for the formulation of agrochemical active substances. Examples which may be mentioned are dichlorophene and benzyl alcohol hemiformal.

Possible humectant components and antifreeze agents which can be used for formulating the powders according to the invention are all those materials which can be employed for such purposes in agrochemical compositions. Substances which can preferably be used are polyhydric alcohols such as glycerol, ethanediol, propanediol and polyethylene glycols of various molecular weights.

Suitable secondary thickeners which can be used for formulating the powders according to the invention are all substances which can be employed for such purposes in agrochemical compositions. Possible substances are, preferably, cellulose derivatives, acrylic acid derivatives, xanthan, modified clays and highly dispersed silica.

Possible solvents which can be used for formulating the powders according to the invention are all organic solvents which can be employed in agrochemical compositions. Substances which are preferably suitable are ketones such as methyl isobutyl ketone and cylohexanone, furthermore amides, such as dimethylformamide, moreover cyclic compounds such as N-methyl-pyrrolidone, N-octyl-pyrrolidone, N-dodecyl-pyrrolidone, N-octyl-caprolactam, N-dodecyl-caprolactam and γ-butyrolactone, in addition strongly polar solvents such as dimethylsulfoxide, furthermore aromatic hydrocarbons such as xylene, or else esters such as propylene glycol monomethyl ether acetate, dibutyl adipate, hexyl acetate, heptyl acetate, tri-n-butyl citrate, diethyl phthalate and di-n-butyl phthalate, and, moreover, alcohols such as ethanol, n- and i-propanol, n- and i-butanol, n- and i-amyl alcohol, benzyl alcohol and 1-methoxy-2-propanol. Water may also be employed as diluent.

If it is intended to prepare seeddressing products, stickers may also be employed for formulating the powders according to the invention. Suitable materials are all customary binders which can be employed in seed-dressing products. Materials which may preferably be mentioned are polyvinylpyrrolidone, polyvinyl acetate, polyvinyl alcohol and tylose.

Especially preferred as stickers are also dispersions of biodegradable polyester/polyurethane/polyureas in water. Such dispersions are known (cf. WO 01/17347).

The powder formulations according to the invention, either as such or else after mixing with further formulation auxiliaries and/or plant treatment agents and, if appropriate, after further dilution with water, can be employed in practice. They are applied by customary methods, that is to say for example by broadcasting, pouring, spraying or atomizing.

It is especially advantageous to convert the powders according to the invention, by adding suitable formulation auxiliaries and, if appropriate, diluents, into seed-dressing products with which a wide range of seed can be treated. Thus, such seed-dressing products are suitable for dressing the seed of cereals such as wheat, barley, rye, oats and triticale, and of the seed of rice, maize, oilseed rape, peas, field beans, cotton, sunflowers and beet, or else the widest possible range of vegetable seed. The seed-dressing product formulations may also be employed for dressing the seed of transgenic plants. In this context, synergistic effects may be observed in combination with the substances formed by expression.

Suitable mixing apparatus for treating the seed with the seed-dressing product formulations are all those which can conventionally be employed for the treatment of seed. Specifically, seed-dressing is done by introducing the seed into a mixer, adding the desired amount of seed-dressing product formulations either as such or after previously diluting them with water, and mixing until the formulation is distributed uniformly on the seed. If appropriate, this is followed by a drying process.

The powder formulations according to the invention and the formulations which can be prepared. therefrom by further mixing with formulation auxiliaries and/or plant treatment compositions are outstandingly suitable for applying agrochemical active substances to plants and/or their environment. They ensure that the active components are released over a prolonged period in the specific amount desired.

The rate of application of the powder formulations according to the invention and of the preparations which can be prepared therefrom by further mixing with formulation auxiliaries can be varied within a substantial range. It depends on the agrochemical active substances which are present in each case, their content in the plant treatment compositions, the indication in question, and the field of application.

The preparation and the use of the powder formulations according to the invention is illustrated by the examples which follow.

### EXAMPLES

### Example 1

A stirred three-necked flask apparatus is charged with 37.82 g of 1,2-propanediol, 100 g of acetone and 0.07 g of dibutyltin dilaurate, and 78.11 g of Desmodur® T 80 are added dropwise with cooling in such a way that the internal temperature climbs to 50° C. The isocyanate content is 0.23% 2.5 hours after the dropwise addition has been ended and after afterreaction at 50° C. After addition of 5 g of water and a further 30 minutes afterreaction at 50° C., the solution is free of isocyanate. 29 g of Folicur are introduced, and the acetone is removed by distillation up to an internal temperature of 160° C. The distillation residue is poured onto a Teflon sheet and, after cooling, pulverized in a Braun mixer. After screening over a mesh size of 63 μm, 98 g of product are obtained. According to particle size determination, 50% of the particles are <24 μm. The active substance content according to GLC is 20.1%.

Release of active substance:

In a sealed 500 ml Erlenmeyer flask, 50.0 mg of the powder are stirred at 20° C. in 200 ml of Cipac 500 ppm water. The amount of Folicur is 50.25 mg/l.

Samples are taken from the suspension at the times indicated hereinbelow and filtered through a 0.2 μm microfilter, and the active substance content is determined by means of HPLC.

| Sampling after [h] | Folicur content [mg/l] |
|---|---|
| 1 | 0.33 |
| 6 | 0.48 |
| 24 | 0.63 |
| 48 | 0.85 |
| 168 | 1.68 |
| 336 | 3.96 |
| 504 | 6.12 |
| 696 | 9.42 |
| 840 | 10.6 |
| 1008 | 11.97 |

### Example 2

In a stirred three-necked flask apparatus, 100 g of a polyester of phthalic acid and 1,2-ethanediol with a mean molar mass of 2000 g/mol and an OH content of 1.7% are molten and dehydrated for 30 minutes at 100° C. and 15 mbar. Thereafter, 9.2 g of Desmodur® PU 1806 (diphenylmethane diisocyanate, isomer mixture, Bayer) are added dropwise in the course of 15 minutes and the mixture is stirred for 4.5 hours at 130° C. Thereafter, the mixture is free of isocyanate according to IR spectrum. 27.3 g of imidacloprid are introduced and the internal temperature is raised to 150° C. The melt is poured onto a Teflon sheet and, when cold, pulverized in a Braun mixer and screened to give particles <125 μm. The active substance content found, determined by HPLC, is 19.4%.

Release of active substance:

In a sealed 200 ml Erlenmeyer flask, 52 mg of the powder are stirred at 25° C. in 100 ml of Cipac 500 ppm water. The amount of imidacloprid is 100 mg/Il.

Samples are taken from the suspension at the times indicated hereinbelow and filtered through a 0.2 μm microfilter, and the active substance content is determined by means of HPLC.

| Sampling after [h] | Imidacloprid content [mg/l] |
|---|---|
| 1 | 12.8 |
| 6 | 30.7 |
| 24 | 74.5 |
| 48 | 125.3 |
| 168 | 213.9 |
| 336 | 252.7 |

### Example 3

In a stirred three-necked flask apparatus, a prepolymer is prepared by introducing 111 g of isophorone diisocyanate and adding 35.4 g of 1,6-hexanediol portionwise at 120° C. Stirring is continued until the calculated value of 11.48% of NCO is reached, or almost reached. 10.92% of NCO were found.

36.6 g of the prepolymer are dissolved in 120 g of methylene chloride, and the solution is added dropwise with cooling at room temperature to a solution of 10.7 benzylamine and 50 g of methylene chloride. A few minutes after the dropwise addition has ended, the solution is free of NCO according to IR spectrum. 21.6 g of imidacloprid are introduced. The mixture is heated slowly to 150° C., during which process methylene chloride is removed by distillation. At 150° C., a vacuum of 15 mbar is applied. The melt is poured onto a Teflon sheet and pulverized in a Braun mixer and the powder is screened to give particles <125 μm. 30.0% of imidacloprid, as determined by HPLC, were found.

Release of active substance:

In a sealed 2 l Erlenmeyer flask, 3333 mg of the powder are stirred at 25° C. in 1000 ml of Cipac 500 ppm water. The amount of imidacloprid is 1000 mg/l.

Samples are taken from the suspension at the times indicated hereinbelow and filtered through a 0.2 μm microfilter, and the active substance content is determined by means of HPLC.

| Sampling after [h] | Imidacloprid content [mg/l] |
|---|---|
| 1 | 13.65 |
| 6 | 26.06 |
| 24 | 59.71 |
| 48 | 99.64 |
| 168 | 333.6 |

### Example 4

A stirred three-necked flask apparatus is charged with 11.8 g of 1,6-hexanediol and 2.16 g of benzyl alcohol, and 24.2 g of isophorone diisocyanate are added dropwise at 90° C. in the course of 35 minutes. Thereafter, the internal temperature is raised to 160° C. in the course of 2.5 hours. The product is NCO-free according to IR spectrum. 16.45 g of imidacloprid are slowly introduced into the melt. The homogeneous melt is poured onto a Teflon sheet and, when cold, conuninuted in a Braun mixer and the powder is screened to give particles <125 μm. 29.9% of imidacloprid, as determined by HPLC, were found.

Release of active substance:

In a sealed 2 l Erlenmeyer flask, 3345 mg of the powder are stirred at 25° C. in 1000 ml of Cipac 500 ppm water. The amount of imidacloprid is 1000 mg/l.

Samples are taken from the suspension at the times indicated hereinbelow and filtered through a 0.2 μm microfilter, and the active substance content is determined by means of HPLC.

| Sampling after [h] | Imidacloprid content [mg/l] |
|---|---|
| 1 | 11.58 |
| 6 | 16.26 |
| 24 | 33.33 |
| 48 | 54.17 |
| 168 | 134.6 |

### Example 5

### Preparation of a Low-Molecular-Weight Polyurethane with Good Crystallization Properties

A stirred three-necked flask apparatus is charged with 168 g of hexamethylene diisocyanate and 46 g of ethanol are added dropwise at 90° C. in the course of 1 hour. Then, 45 g of 1,4-butanediol are added dropwise in the course of a further hour, and the temperature is raised to 160° C. Stirring is continued at this temperature until the melt no longer contains isocyanate according to IR spectroscopy. The product is poured onto a Teflon sheet and, when cold, comminuted in a Braun mixer.

### Example 6

### Preparation of a Polyurethane in Acetonic Solution

A stirred three-necked flask apparatus is charged with 35.3 g of 1,2-propanediol and 71.5 g of acetone, and 64.8 g of isomer mixture consisting to 80% of toluylene diisocyanate and to 20% of 2,6-toluylene diisocyanate are added dropwise, with cooling. Stirring is continued for 2 hours at 50° C. The isocyanate content of the clear solution is 0.1%.

### Example 7

### Preparation of a Powder Formulation with a Temperature-Sensitive Active Substance

In a stirred three-necked flask apparatus, 30 g of methiocarb, 20 g of polyurethane of Example 5, 85.8 g of the acetonic solution of Example 6 and 0.1 g of stearic acid are heated to 110° C., and the acetone is removed via a distillation head, first under atmospheric pressure and thereafter at up to 15 mbar. The melt is poured onto a Teflon sheet and, when cold, ground in a Braun mixer and screened to give particles <125 μm. The active substance content identified by HPLC is 26.5%.

Release of active substance:

In a sealed 500 ml Erlenmeyer flask, 0.038 g of the powder is stirred in 200 ml of buffer solution pH 4 (Riedel-de Haen, Art. No. 33543) at 25° C. The amount of methiocarb is 50 mg/l.

Samples are taken from the suspension at the points in time indicated hereinbelow and filtered through a 0.2 μm microfilter, and the active substance content is determined by means of HPLC.

| Sampling after [h] | Methiocarb content [mg/l] |
|---|---|
| 1 | 4.35 |
| 6 | 7.16 |
| 24 | 10.13 |
| 48 | 11.83 |

1. A powder comprising,
at least one agrochemical active substance,
at least one polyurethane, or polyurethane urea, or a mixture thereof, and,
optionally, one or more additives,
wherein said powder has a particle size of less than 125 μm in diameter.

2. The powder according to claim 1, wherein the polyurethane, or the polyurethane urea, has a mean molar mass of from 200 to 50,000 g/mol.

3. The powder according to claim 2, wherein the polyurethane, or the polyurethane urea, has a mean molar mass of from 250 to 20,000 g/mol.

4. The powder according to claim 1, wherein,
the agrochemical active substance is between 1 and 50% by weight,
the polyurethane, the polyurethane urea, or the mixture thereof is between 50 and 99% by weight, and
the additive is between 0 and 30% by weight.

5. The powder according to claim 1, wherein the agrochemical active substance is selected from a group consisting of imidacloprid, carpropamid, tebuconazole and methiocarb.

6. A process for the preparation of a powder according to claim 1, comprising,
homogenizing
at least one agrochemical active substance,
at least one polyurethane, or polyurethane urea, or a mixture thereof, and,
optionally, one or more additives,
in a melt at a temperature between 50° C. and 200° C., and,
cooling and comminuting the mixture to obtain a powder having a particle size of less than 125 μm in diameter.

7. The process according to claim 6, wherein the agrochemical active substance is mixed with the polyurethane, the polyurethane urea, or the mixture thereof, in the presence of a solvent and the solvent is subsequently removed.

8. A method of applying an agrochemical active substance to plants or their environment, comprising contacting plants or their environment with a powder according to claim 1.

9. A composition according to claim 1 further comprising one or more extenders, or surface-active agents.

10. The composition according to claim 9, wherein the agrochemical active substance is selected from a group consisting of imidacloprid, carpropamid, tebuconazole and methiocarb.

11. The composition according to claim 9, wherein the polyurethane, or the polyurethane urea, has a mean molar mass of from 200 to 50,000 g/mol.

12. The composition according to claim 11, wherein said polyurethane, or said polyurethane urea, has a mean molar mass of from 200 to 20,000 g/mol.

13. The composition according to claim 9, wherein,
the agrochemical active substance is between 1 and 50% by weight,
the polyurethane, the polyurethane urea, or the mixture thereof is between 50 and 99% by weight, and
the additive is between 0 and 30% by weight.

14. The powder according to claim 4, wherein the agrochemical active substance is selected from a group consisting of imidacloprid, carpropamid, tebuconazole and methiocarb.

15. The powder according to claim 4, wherein the polyurethane, or the polyurethane urea has a mean molar mass of from 200 to 50,000 g/mol.

16. The powder according to claim 15, wherein the polyurethane, or the polyurethane urea has a mean molar mass of from 200 to 20,000 g/mol.
